(19) United States
(12) Patent Application Publication
(10) Pub. No.: US 2012/0101534 A1
(43) Pub. Date: Apr. 26, 2012

(54) Bone Screw For Orthopedic Apparatus

(76) Inventor: Richard B. Pitbladdo, Naples, FL (US)

(21) Appl. No.: 13/336,614

(22) Filed: Dec. 23, 2011

Related U.S. Application Data
(63) Continuation-in-part of application No. 12/170,950, filed on Jul. 10, 2008.
(60) Provisional application No. 61/508,191, filed on Jul. 15, 2011.
(60) Provisional application No. 60/949,596, filed on Jul. 13, 2007.

(51) Int. Cl. A61B 17/86

(52) U.S. Cl. 606/309

(57) ABSTRACT

A bone screw deforms on the same order as the bone, thus providing substantially uniform loading along an entire length of the thread of the bone screw. The bone screw evenly distributes stress by matching the effective cross-sectional area of the bone screw times its modulus of elasticity with the effective cross-sectional area of the parent material (i.e. bone) times its modulus of elasticity so that so that the linear deformation of each is preferably substantially equal to the other.

### REFERENCE TO RELATED APPLICATIONS

This application claims one or more inventions which were disclosed in Provisional Application No. 61/508,191, filed Jul. 15, 2011, entitled “BONE SCREW FOR ORTHOPEDIC APPARATUS”.

This is also a continuation-in-part application of copending application Ser. No. 12/170,950, filed Jul. 10, 2008, entitled “BONE SCREW FOR ORTHOPEDIC APPARATUS”, which claimed priority from Provisional Application No. 60/949,596, filed Jul. 13, 2007, entitled “BONE SCREW FOR ORTHOPEDIC APPARATUS”.

The benefit under 35 USC §119(e) of the United States provisional applications is hereby claimed, and the aforementioned applications are hereby incorporated herein by reference.

### BACKGROUND OF THE INVENTION

1. Field of the Invention

The invention pertains generally to the design of screws to secure orthopedic reinforcements to bones.

2. Description of Related Art

The attachments of orthopedic reinforcements to bone are limited by how well the screws inserted into the bone secure the reinforcements. It has been determined that the attachment strength of a screw with a few (2-3) threads is substantially as strong as a screw with many (4 or more) threads. Using a longer standard screw versus a short standard screw makes little difference in the attachment strength. This is a major drawback of present practice in the applications where attachment strength is important.

### SUMMARY OF THE INVENTION

The present invention provides bone screw designs whereby the screw deforms on the same order as the bone, thus providing substantially uniform loading along a substantial portion of the length of a thread of the bone screw. The lengthwise tension loading is much more uniform than the tension loading in the prior art. In fact, some of the embodiments of the present invention provide substantially uniform loading along the entire length of the thread.

A bone screw of the present invention evenly distributes stress by matching the effective cross-sectional area of the bone screw times its modulus of elasticity with the effective cross-sectional area of the parent material (i.e. bone) times its modulus of elasticity so that they are preferably substantially equal to each other.

### BRIEF DESCRIPTION OF THE DRAWINGS

FIG. 1 shows a prior art bone screw.

FIG. 2 shows a bone screw with a hollow center in an embodiment of the present invention.

FIG. 3 shows a helical bone screw in an embodiment of the present invention.

FIG. 4 shows a bone screw with a tapered hollow center in an embodiment of the present invention.

FIG. 5 shows a tapered helical bone screw in an embodiment of the present invention.

FIG. 6 shows a tapered insert for the tapered helical bone screw of FIG. 5 in an embodiment of the present invention.

FIG. 7 shows a helical bone screw with a center reinforcement insert in an embodiment of the present invention.

FIG. 8A shows a tapered insert for the tapered helical bone screw of FIG. 7 in an embodiment of the present invention.

FIG. 8B shows a tapered insert for the tapered helical bone screw of FIG. 7 in another embodiment of the present invention.

FIG. 8C shows a tapered insert for the tapered helical bone screw of FIG. 7 in another embodiment of the present invention.

FIG. 9 shows a bone screw with a hollow center over a portion of its length in an embodiment of the present invention.

FIG. 10 shows a bone screw with the thread attached to the shank only at the ends of the thread in an embodiment of the present invention.

FIG. 11 shows a bone screw with the thread attached to the shank only at the distal end of the thread in an embodiment of the present invention.

FIG. 12 shows a bone screw with the thread attached to the shank only at the head end of the thread in an embodiment of the present invention.

FIG. 13 shows a bone screw with a flexible thread in an embodiment of the present invention.

FIG. 14 shows a bone screw with a tapered shank at the head end and a flexible thread in an embodiment of the present invention.

FIG. 15 shows a bone screw with a tapered shank and a flexible thread in an embodiment of the present invention.

FIG. 16 shows a bone screw with a flexible thread with the thread attached to the shank only at the ends of the thread in an embodiment of the present invention.

FIG. 17 shows the relative deformation of flexible threads of a bone screw in a bone in an embodiment of the present invention.

FIG. 18 shows the bone screw of FIG. 17 attaching an appliance to bone.

FIG. 19 shows a bone screw with a constant cross section attaching an appliance to bone.

### DETAILED DESCRIPTION OF THE INVENTION

A standard screw made of stainless steel or titanium is much more rigid than the bone into which it is inserted. When a lengthwise/longitudinal tension load is applied to the screw, the bone deforms much more readily than the screw. The loading of the bone screw is not at all uniform along a length of the thread. Thus, only a few sections of the thread of the screw support the entire load. If the load on the bone is too high at these sections, the bone yields to failure at these sections and transfers the stress to other sections of the thread. Thus, there is a cascading phenomenon until the bone at all sections of the thread fails and the attachment is broken.

The present invention includes bone screws with different configurations in various embodiments. There are two different groups of embodiments. In one group of embodiments, the shape of the bone screw is varied to decrease the total cross-sectional area of the bone screw. In another group of embodiments, the material from which the bone screw is made has a much lower modulus of elasticity than titanium or stainless steel, the materials currently used for bone screws. The shape embodiments use screws preferably made from conventional materials (such as stainless steel or titanium), whereas the material embodiments require a material with mechanical properties matched to the bone material by the relationship discussed herein. Both groups of embodiments evenly distribute stress by matching the effective cross-sectional area of the screw times its modulus of elasticity with the effective cross-sectional area of the parent material (i.e. bone) times its modulus of elasticity so that they are preferably substantially equal to each other.

Stress analysis of a screw inserted into a parent material shows that the screw may be made from a material which is substantially stiffer than the parent material. To create a relatively even distribution of stress from the screw threads to the parent material, the present invention substantially equates the effective cross-sectional area of the screw (Aₛ) times its modulus of elasticity (Eₛ) to the effective cross-sectional area of the parent material (Aₚ) times its modulus of elasticity (Eₚ).

(Aₛ)×(Eₛ)≈(Aᵣ)×(Eₚ). (1)

This relationship equates equal and opposite forces on the screw threads along the length of the attachment. In the embodiments where all or a portion of the screw resembles a spring, the force on the screw threads can be expressed as the spring rate (Kₛ) of the thread times the length (lₛ) of the thread.

(Kₛ)×(lₛ)≈(Aₚ)×(Eₚ) (2)

Note that:

(Aₛ)=(Kₛ)×(lₛ)/(Eₛ). (3)

In a preferred embodiment of the present invention, the parent material is bone.

As defined herein, the effective cross-sectional area of the bone is the cross-sectional area which is substantially deformed or displaced under load by the inserted screw. The effective cross-sectional area of the portion of the screw with a shank is either the actual cross-sectional area of the screw material when you take a cross-section perpendicular to the length of the screw at the shank or a function of the spring rate of the screw as shown in equation (3) above. The effective cross-sectional area of the portion of the screw including only one or more threads (and no shank) is a function of the spring rate of the thread as shown in equation (3) above.

In preferred embodiments of the present invention, for at least one point or section along the length of the bone screw, the effective cross-sectional area of the bone screw material times the modulus of elasticity of the screw material is substantially equal to the effective cross-sectional area of the bone times the modulus of elasticity of the bone.

In other preferred embodiments of the present invention, for at least one point or section along the length of the bone screw, the spring rate of that section of the bone screw times the length of that section of the bone screw is substantially equal to the effective cross-sectional area of the bone times the modulus of elasticity of the bone.

As an example, if a titanium screw with a modulus of elasticity (Eₛ) of 16.5×10⁶ psi and a cross sectional area (Aₛ) of 0.01 square inches was secured in a bone with an effective area (A_{b}) of 0.25 square inches and a modulus of elasticity (E_{b}) of 0.66×10⁶ psi, the load on the bone material would be substantially evenly distributed (16.5×10^{6×0.01=0.66×10}⁶×0.25) along the entire length of the screw thread.

In preferred embodiments, the effective cross-sectional area of a specific bone structure is determined by mathematical simulation using a technology known as “Finite Element Analysis” (FEA). It would not be practical to determine this during surgery; however, the effective cross-sectional area of various specific bone structures is preferably cataloged via FEA. This preferably creates a catalog of effective cross-sectional areas for different bones and/or different types of bones. In a preferred embodiment, the catalog is preferably compiled from data on different bones including, but not limited to, the femur, the humerus, the tibia, the metacarpals, the metatarsals, the phalanges, vertebrae, the carpals, the tarsals, the scapula, the sternum, the cranium, the os coxae, the pelvis, the ribs, the sacrum, the mandible, and the patella, resulting in the most effective cross-sectional area of each bone. In some embodiments, the FEA catalog may include more general information on different types of bones including, but not limited to, long bones, short bones, flat bones, irregular bones, and sesamoid bones. Using information from this catalog and knowing in advance the bone configuration enables the surgeon to select the appropriate bone screws.

For example, the effective cross sectional area of a vertebra to which an appliance is to be attached would be determined via FEA. In practice, a standard would be established for the attachment of specific appliances to vertebrae with specific bone screw sizes and configurations. These bone screw sizes and configurations would preferably vary to include bone screw selection options based on bone health.

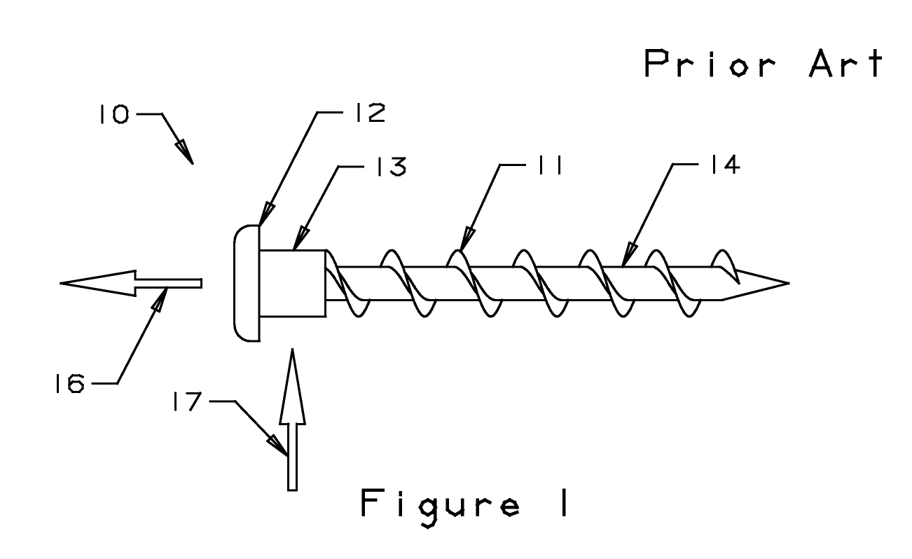

FIG. 1 shows a prior art bone screw (10). It is essentially the same as a standard button head screw. It consists of a thread (11) which is part of the shank (14) of the screw (10). The prior art bone screw (10) has a shoulder (13) where it passes through the orthopedic device to be attached to the bone and a head (12) to secure the orthopedic device to the bone. A lengthwise/longitudinal tension load (16) is applied to the screw when the screw is attached to the bone. The stresses on the attachment of an orthopedic device to a bone also include a shear force (17) perpendicular to the lengthwise/longitudinal axis of the screw. A bone screw of the present invention is preferably designed to target the longitudinal tension load (16); however, bone screws of the present invention may also be designed to have the shear and bending strength to accommodate the shear force (17).

Bone typically has a lower modulus of elasticity than the 0.66×10⁶ noted in the example. Therefore, a bone screw of titanium or stainless steel must have a lower effective cross-sectional area than the cross-sectional area of the prior art screws in order to more evenly distribute the load across the threads of the screw.

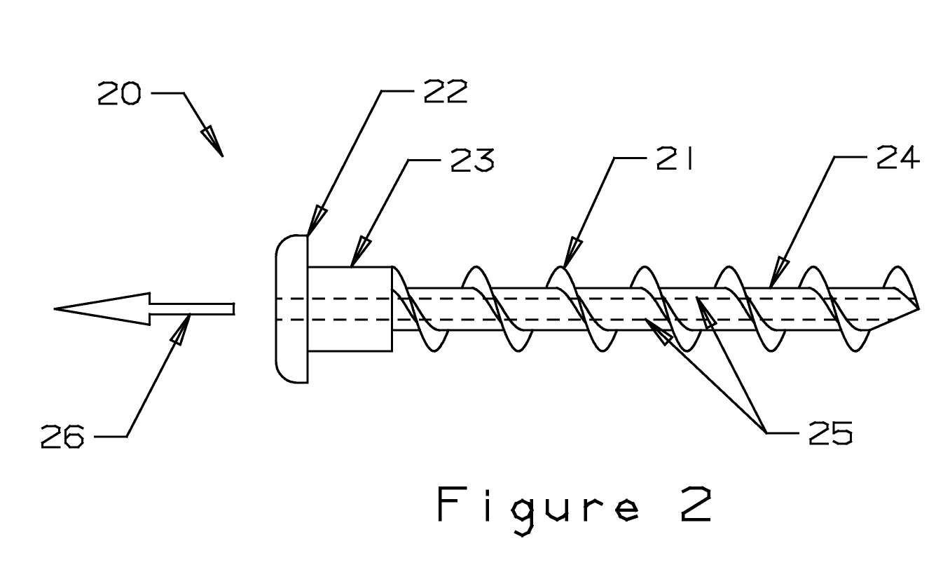

FIG. 2 shows a bone screw (20) in an embodiment of the present invention. The screw (20) has a thread (21), a shank (24), a shoulder (23), and a head (22). The bone screw (20) also has a hole (25) in the centerline of the bone screw, thus reducing its cross-sectional area. The diameter of the hole (25) is chosen to reduce the cross-sectional area of the shank (24) and the thread (21) combined, to match the relationship between the modulus of elasticity and cross-sectional area previously described. In a preferred embodiment, the bone screw (20) may be made of a material that has a high modulus of elasticity, such as titanium or stainless steel. A lengthwise (longitudinal) tension load (26) is applied to the screw when the screw is attached to the bone. In a preferred embodiment, the center cavity is filled with an insert or a paste of artificial bone.

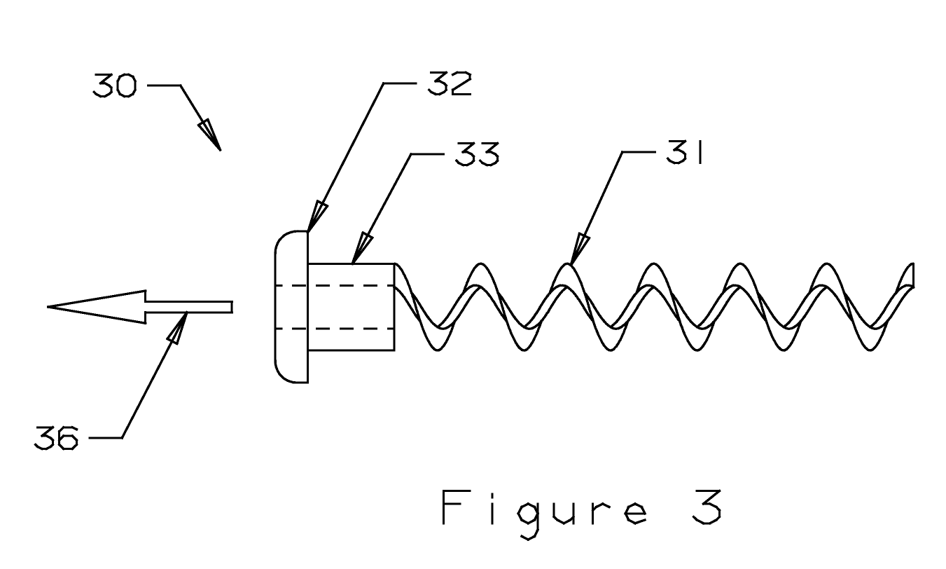

FIG. 3 shows a bone screw (30) in another embodiment of the present invention, which is the limit in reducing the effective cross-sectional area. The screw has a thread (31), a shoulder (33), and a head (32); however, it has no shank. It is in actuality a helical spring. Because the cross-sectional area has been significantly reduced, in a preferred embodiment, the bone screw (30) may be made of a material with a high modulus of elasticity, such as titanium or stainless steel. A lengthwise (longitudinal) tension load (36) is applied to the screw when the screw is attached to the bone.

Because of its flexibility, the helical bone screw (30) requires tapping of the bone prior to installation. A tap of suitable shape would be required. After insertion of the threaded helix, the center cavity is preferably filled with a plug or a paste of artificial bone.

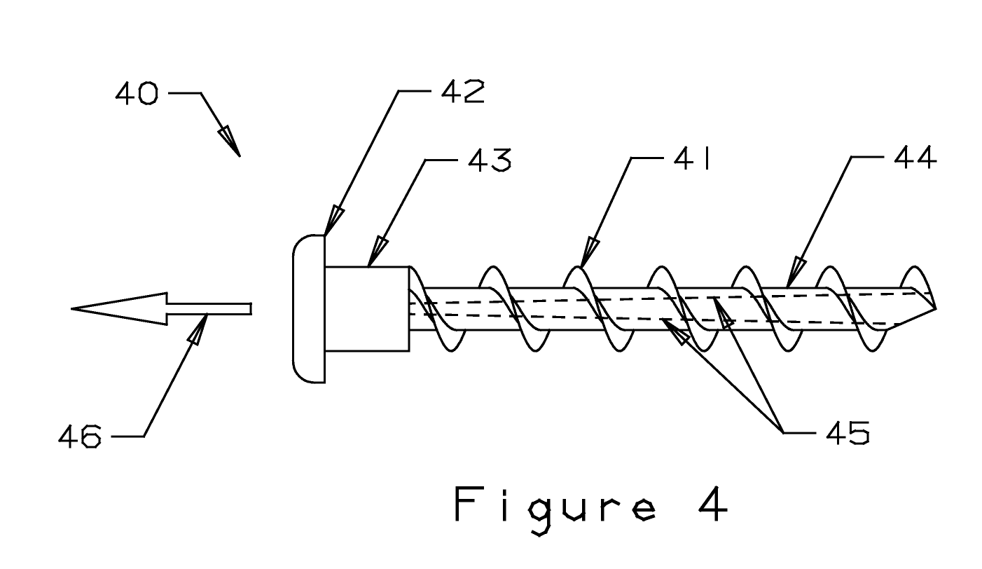

The bone screw configurations of FIGS. 2 and 3 would be ideal if the primary attachment parameter is lengthwise (longitudinal) tension and if it was possible to determine the exact modulus of elasticity of an individual's bone with accuracy. This is not presently possible considering the various effects of age and health on an individual's bone properties. To minimize the need for an exact determination of the bone properties, the embodiments of FIGS. 4, 5, 7, and 9 have a variable cross-section along the length of the bone screw.

FIG. 4 shows a screw (40) in an alternative embodiment of the present invention, which has a variable effective cross-sectional area along its length. The screw (40) has a thread (41), a shank (44), a shoulder (43), and a head (42), which are substantially the same as shown in FIG. 2. The difference between this embodiment and the embodiment shown in FIG. 2 is that the hole (45) in the centerline of the bone screw is tapered, thus varying the cross-sectional area along the length of the screw (40). A lengthwise (longitudinal) tension load (46) is applied to the screw when the screw is attached to the bone. In a preferred embodiment, the bone screw (40) may be made of a material with a high modulus of elasticity, such as titanium or stainless steel. In another preferred embodiment, the center cavity is filled with an insert or a paste of artificial bone.

This embodiment, with its range of cross-sectional area, does not give as strong an attachment as the embodiments of FIGS. 2 and 3 when these embodiments are perfectly matched to the bone elasticity; however, it allows for a large margin of mismatch to the bone elasticity and gives greatly improved stress distribution than the prior art.

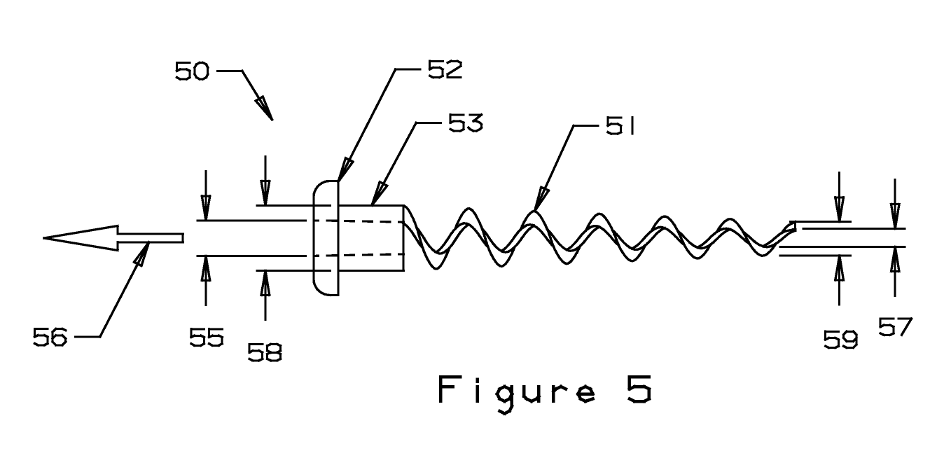

FIG. 5 shows a bone screw (50) in another embodiment of the present invention, which has a variable effective cross-sectional area along its length. The screw (50) has a shoulder (53), a head (52), and a tapered helical thread (51). The inside diameter (55) of the thread (51) at the shoulder (53) end is greater than the inside diameter (57) at the distal end. Additionally, the outside diameter (58) of the thread (51) at the shoulder (53) end is greater than the outside diameter (59) at the distal end. The difference between the outside diameter (58) and the inside diameter (55) at the shoulder (53) end is greater than the difference between the outside diameter (59) and the inside diameter (57) at the distal end. In another embodiment, the outside diameter (58) of the thread (51) at the shoulder (53) end is equal to the outside diameter (59) at the distal end. In this embodiment, the difference between the outside diameter (58) and the inside diameter (55) at the shoulder (53) end is greater than the difference between the outside diameter (59) and the inside diameter (57) at the distal end. The tapered helical shape of the bone screw has the effect of making the effective elastic modulus of the screw as a system substantially lower than a prior art bone screw. The taper may be in the thickness and/or the shape of the thread (51). In a preferred embodiment, the bone screw (50) may be made of a material with a high modulus of elasticity, such as titanium or stainless steel. A lengthwise (longitudinal) tension load (56) is applied to the screw when the screw is attached to the bone.

The tapered helical bone screw (50) shown in FIG. 5 requires tapping of the bone prior to installation. A tap of suitable shape would be required.

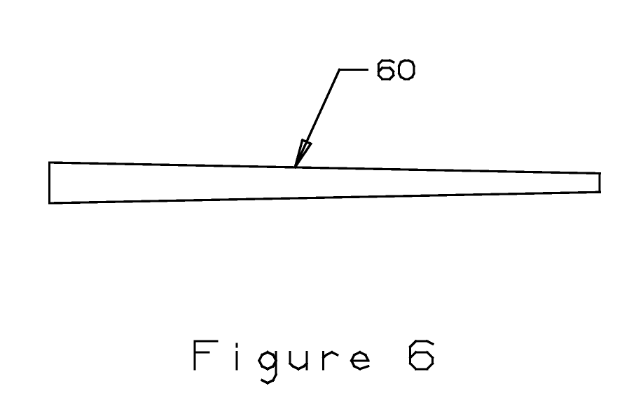

FIG. 6 shows a tapered insert (60) that is preferably inserted into the center of the tapered helical bone screw (50) shown in FIG. 5. The tapered insert (60) is preferably made of material that is compatible with bone, i.e. artificial bone. The tapered insert (60) maintains the threads in position until the bone has healed.

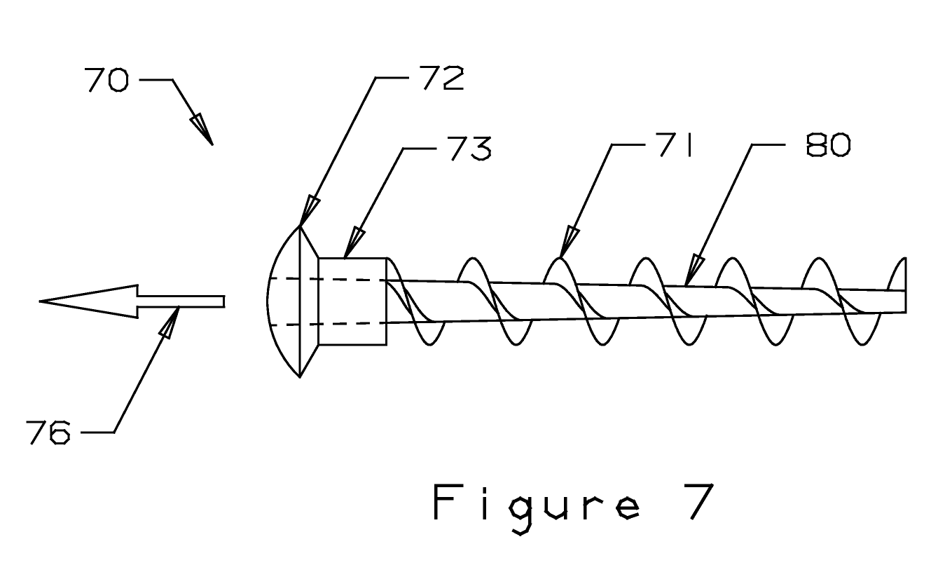

FIG. 7 shows a bone screw (70) in another embodiment of the present invention, which has a variable effective cross-sectional area along its length. The screw (70) has a shoulder (73), a head (72), and a helical shape of the thread (71). The head (72) shown in FIG. 7 is a countersunk head. A lengthwise (longitudinal) tension load (76) is applied to the screw when the screw is attached to the bone. A tapered center insert (80) is inserted into the center of the helical shaped thread (71). In a preferred embodiment, the bone screw (70) may be made of a material with a high modulus of elasticity, such as titanium or stainless steel.

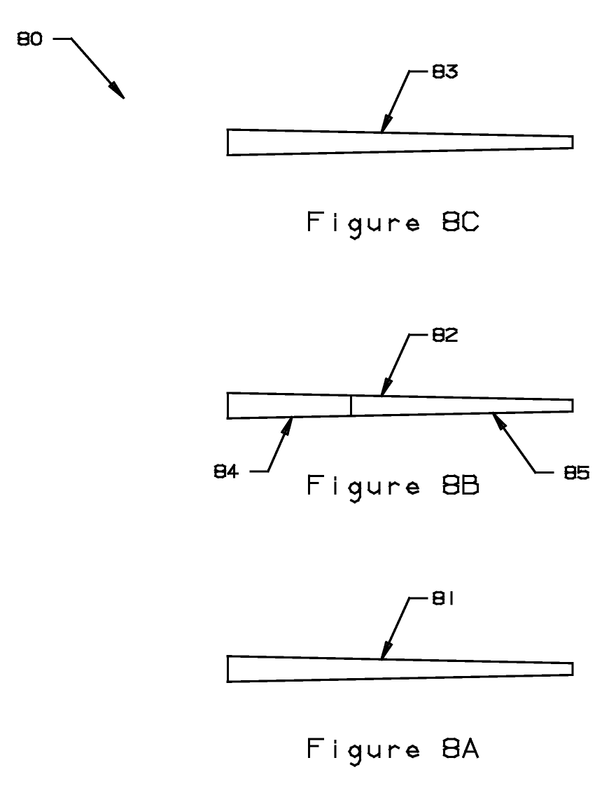

FIGS. 8A through 8C show three configurations of a tapered insert (80) that is preferably inserted into the center of the tapered helical bone screw (70) shown in FIG. 7. The tapered insert (81) shown in FIG. 8A is preferably made from a material with a high modulus of elasticity, such as titanium or stainless steel. The tapered insert (82) shown in FIG. 8B is made from two materials. The head end (84) of the insert (82) is made from a material with a high modulus of elasticity, such as titanium or stainless steel, and the distal end (85) is made from artificial bone material. The inserts (81) and (82) act in the same manner as the shank of the prior art bone screw (10) by providing an increase in the shear and bending strength of the bone screw; however, these inserts (81) and (82) do not change its longitudinal elasticity. The tapered insert (83) shown in FIG. 8C is made from artificial bone material. All of the configurations (81), (82) and (83) of the tapered insert (80) maintain the threads in position until the bone has healed. All of these configurations could alternatively be used as inserts for the bone screw shown in FIG. 5.

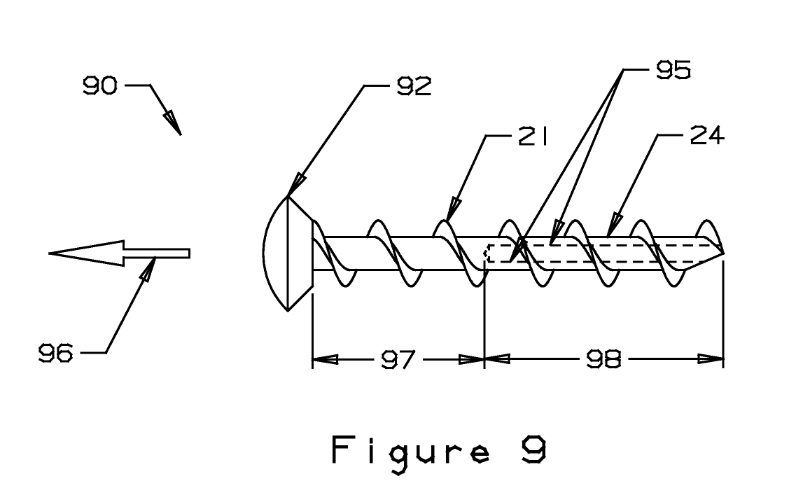

FIG. 9 shows an embodiment similar to the embodiment shown in FIG. 2, except that the center hole (95) has a depth (98) that does not extend the entire length of the screw (90) and the head configuration is different. A lengthwise (longitudinal) tension load (96) is applied to the screw when the screw is attached to the bone. In this embodiment, the bone screw (90) has substantially the same shear and bending strength as the screw (10) of the prior art because of the length of the solid portion (97) of the shank (24). This embodiment also has the advantages of the lengthwise tensile properties of the present invention because of the lower cross-sectional area of the shank (24) section (98) containing the hole (95). The head (92) configuration has no shoulder and is countersunk to match the orthopedic apparatus. In another preferred embodiment, the center cavity is filled with an insert or a paste of artificial bone.

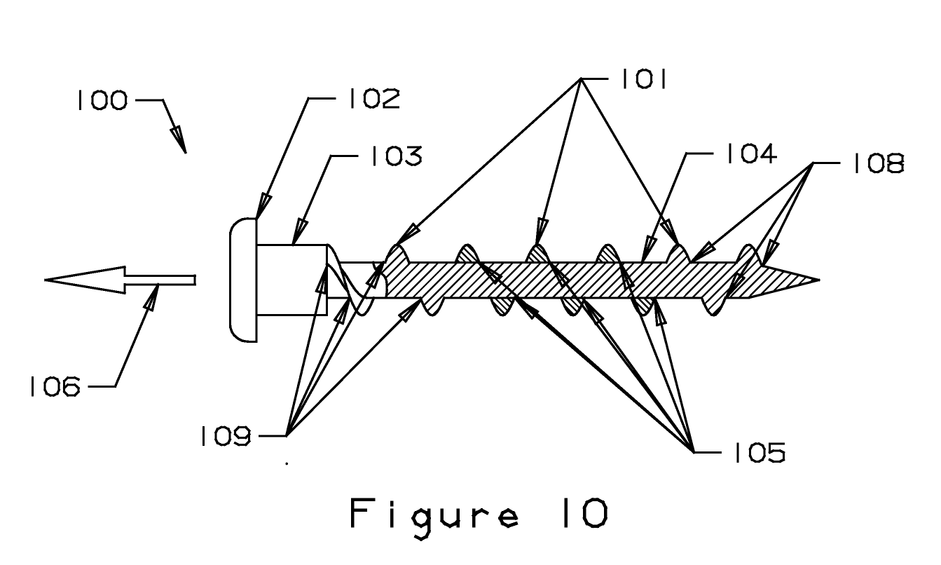

FIG. 10 shows an embodiment of a bone screw (100), where the thread (101) is attached to the shank (104) at only the head end and the distal end of the shank. While the configuration shown in FIG. 10 includes a button head (102) and a shoulder (103), other head configurations are also possible. The cross-hatched areas in FIG. 10 are a section through the center of the screw (100). The thread (101) is attached to the shank (104) at locations (108) at the distal end of the screw (100) and at locations (109) at the head end of the screw (100). The central section of the thread (101) is free to deform longitudinally relative to the shank at the sliding surfaces (105). The central section of the bone screw thread (101) deforms longitudinally to distribute the load on the thread (101) more evenly when a lengthwise (longitudinal) tension load (106) is applied to the screw (100). In this embodiment, the bone screw (100) has substantially the same shear and bending strength as the screw (10) of the prior art because of the length of the solid shank (104).

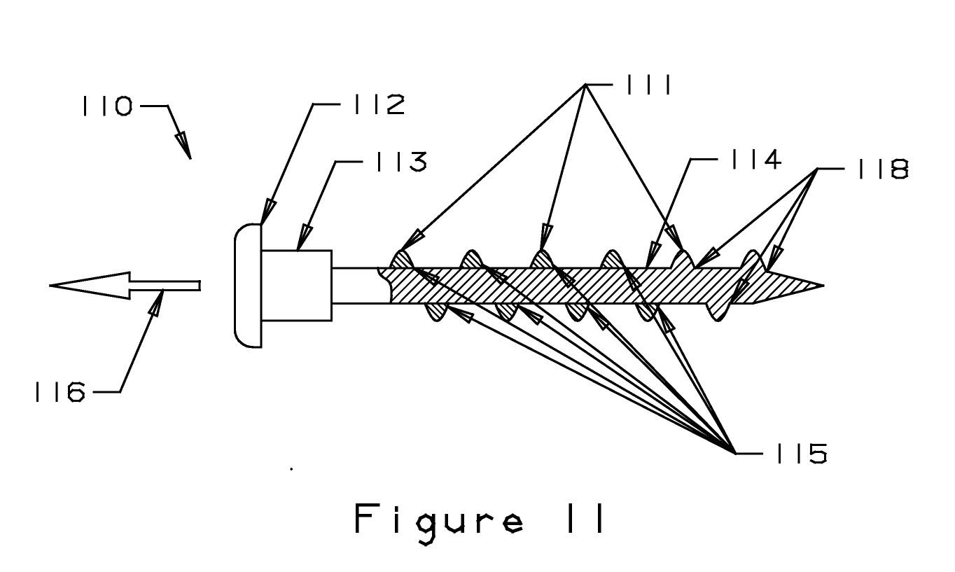

FIG. 11 shows an embodiment similar to the embodiment in FIG. 10, except that the thread (111) is attached to the shank (114) at only the distal end of the shank (114). The bone screw (110) in FIG. 11 includes a head (112) and a shoulder (113), although other head configurations could alternatively be used. The cross-hatched areas in FIG. 11 are a section through the center of the screw (110). The thread (111) is attached to the shank (114) at locations (118) at the distal end of the screw (110). The portion of the thread (111) not at the distal end is free to deform longitudinally relative to the shank at the sliding surfaces (115). In this embodiment, the head end of the bone screw thread (111) deforms longitudinally to distribute the load on the thread (111) more evenly when a lengthwise (longitudinal) tension load (116) is applied to the screw (110). The bone screw (110) has substantially the same shear and bending strength as the screw (10) of the prior art because of the length of the solid shank (114).

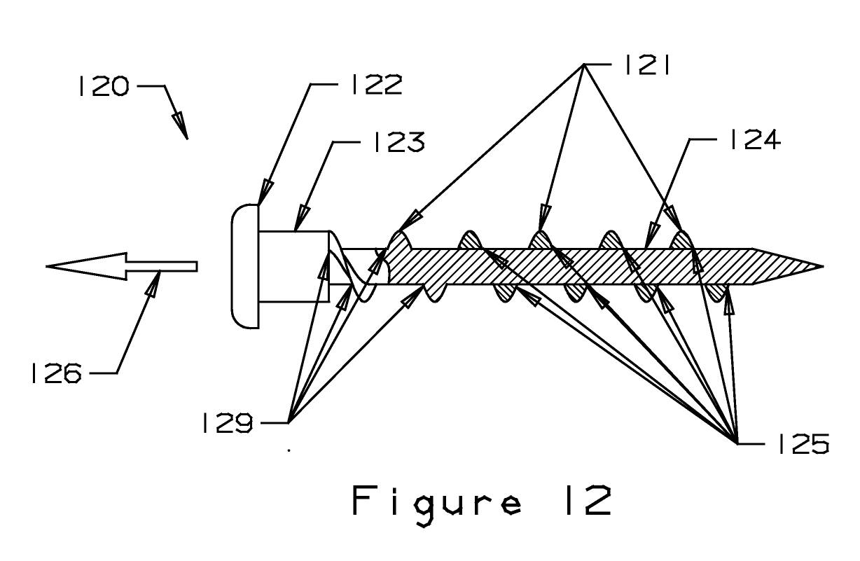

FIG. 12 shows another embodiment of a bone screw (120), where the thread (121) is attached to the shank (124) at only the end of the shank (124) closest to the head (122) and the shoulder (123). The cross-hatched areas in FIG. 12 are a section through the center of the screw (120). The thread (121) is attached to the shank (124) at locations (129) at the head end of the screw (120). The portion of the thread (121) at the distal end of the screw (120) is free to deform longitudinally relative to the shank at the sliding surfaces (125). The distal section of the bone screw thread (121) deforms longitudinally to distribute the load on the thread (121) more evenly when a lengthwise (longitudinal) tension load (126) is applied to the screw (120). In this embodiment, the bone screw (120) has substantially the same shear and bending strength as the screw (10) of the prior art because of the length of the solid shank (124).

Comparing the embodiments of FIGS. 10, 11, and 12, the embodiment shown in FIG. 12 would withstand a higher tension load (126) than the tension loads (106) and (116) in the embodiments of FIGS. 10 and 11. This is because the bone failure normally starts toward the distal end of the bone screw. The embodiment in FIG. 12 would require tapping the bone prior to insertion, whereas the embodiments of FIGS. 10 and 11 could be self-tapping.

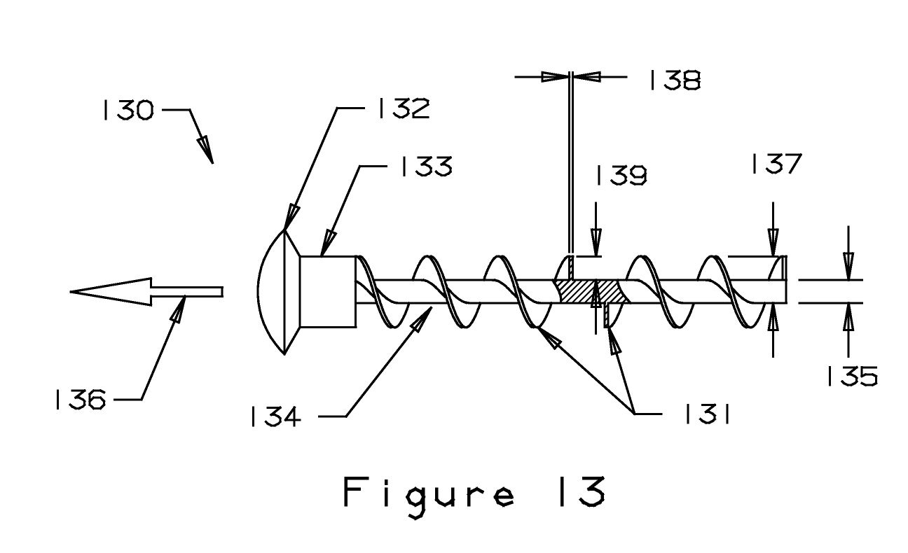

FIG. 13 shows a bone screw (130) in another embodiment of the present invention. The bone screw (130) in this embodiment has a shank (134) with a relatively small diameter (135) compared with the diameter (137) of the thread (131). The screw (130) has a head (132), a shoulder (133), a shank (134), and a helical shape of the thread (131). The head (132) shown in FIG. 13 is a countersunk head. A lengthwise (longitudinal) tension load (136) is applied to the screw when the screw is attached to the bone. The thread (131) has a rectangular cross-section with a height (139) and thickness (138). The thickness (138) of the thread (131) is substantially less than its height (139) such that the thread is flexible and can deform to more uniformly distribute the tension load (136) into the bone in which it is inserted. In a preferred embodiment, the bone screw (130) may be made of a material with a high modulus of elasticity, such as titanium or stainless steel.

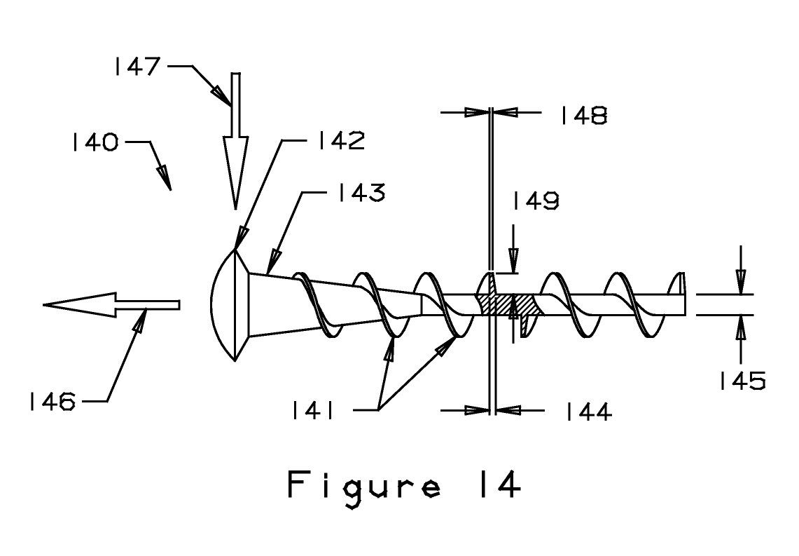

FIG. 14 shows another embodiment of a bone screw (140), which has a shank with a relatively small diameter (145) compared with the diameter of the thread (141). The bone screw (140) has a head (142), a tapered shank (143) at the head end, and a helical shape of the thread (141). The tapered shank reduces the bending strain due to the shear force (147). A lengthwise (longitudinal) tension load (146) is applied to the screw when the screw is attached to the bone. The thread (141) has a trapezoidal cross-section with a height (149), a base thickness (144) and a peripheral thickness (148). Like the embodiment in FIG. 13, the thickness (varying from 148 to 144) of the thread (141) is substantially less than its height (149) such that the thread is flexible and can deform to more uniformly distribute the tension load (146) into the bone in which it is inserted. In a preferred embodiment, the bone screw (140) may be made of a material with a high modulus of elasticity, such as titanium or stainless steel.

The rectangular cross-section of the thread (131) in FIG. 13 and the trapezoidal cross-section of thread (141) in FIG. 14 represent only two of many cross-sections which may be used in embodiments of this invention. The important parameter is that the thread have a height (139 and 149) which is substantially greater than the thread thickness (138, 144 and 148) such that the thread can deform to more uniformly distribute tension load into the bone.

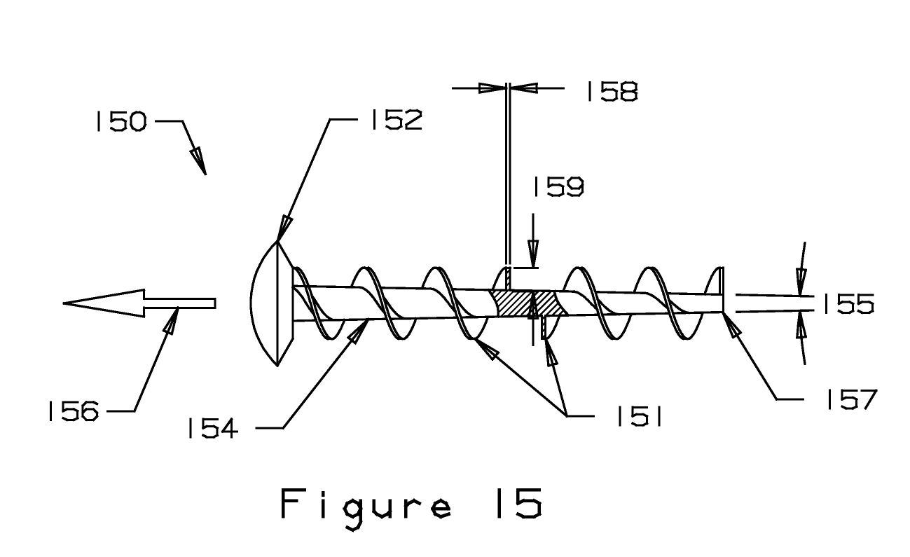

FIG. 15 shows another embodiment of a bone screw (150) with a flexible thread. The bone screw (150) has a head (152), a shank (154) which is tapered, an included acute angle (155) over its entire length, and a helical shape of the thread (151). A lengthwise (longitudinal) tension load (156) is applied to the screw when the screw is attached to the bone. The tapered shank provides greater longitudinal deformation at the distal end (157) than at the head end. Like the embodiment in FIG. 13, the thickness (158) of the thread (151) is substantially less than its height (159) such that the thread is flexible and can deform to more uniformly distribute the tension load (156) in the bone into which it is inserted. In a preferred embodiment, the bone screw (150) may be made of a material with a high modulus of elasticity, such as titanium or stainless steel.

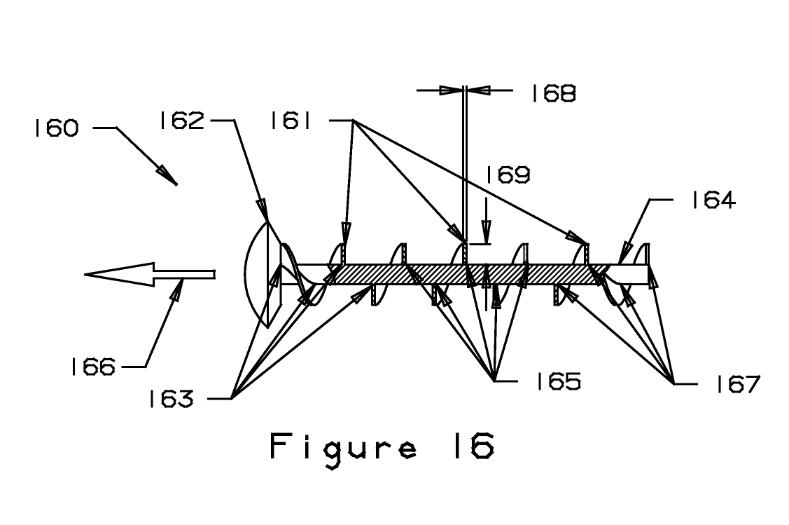

FIG. 16 shows an embodiment of a bone screw (160) with a flexible thread (161) where the thread (161) is attached to the shank (164) at only the head end and the distal end of the shank (164). The bone screw (160) has a head (162), a shank (164), and a helical shape of the thread (161). A lengthwise (longitudinal) tension load (166) is applied to the bone screw when the bone screw is attached to the bone. Like the embodiment in FIG. 13, the thickness (168) of the thread (161) is substantially less than its height (169) such that the thread is flexible and can deform to more uniformly distribute the tension load (166) in the bone into which it is inserted. The thread (161) is attached to the shank (164) at locations (167) at the distal end of the screw (160) and at locations (163) at the head end of the screw (160). The central section of the thread (161) is free to deform longitudinally relative to the shank at the sliding surfaces (165). The central section of the bone screw thread (161) deforms longitudinally to distribute the load on the thread (161) more evenly when a lengthwise (longitudinal) tension load (166) is applied to the screw (160). In a preferred embodiment, the bone screw (160) may be made of a material with a high modulus of elasticity, such as titanium or stainless steel.

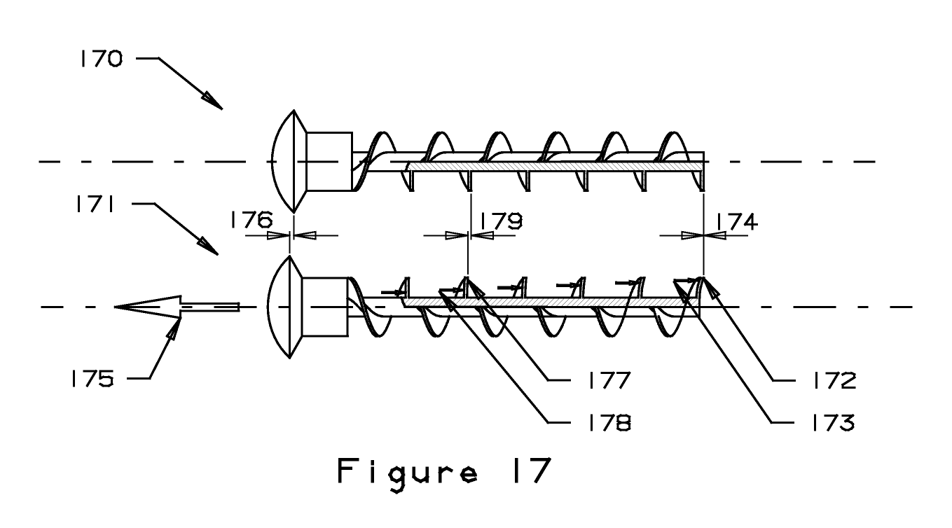

FIG. 17 shows the relative horizontal motion (176) of a stiff bone screw (170) with flexible threads (172 and 177) in a substantially less stiff bone. Bone screw (170) represents the horizontal position of an unloaded bone screw, whereas bone screw (171) represents the horizontal position (relative horizontal motion) and deformation of the flexible threads (172 and 177) of a bone screw under load (175). The head of bone screw (171) translates a horizontally distance (176), whereas the tip of thread (172) under load (173) deforms such that it translates essentially zero (174). Correspondingly the tip of thread (177) translates horizontally distance (179) under load (178). Distance (179) is less than distance (176) because of the deformation of thread (177).

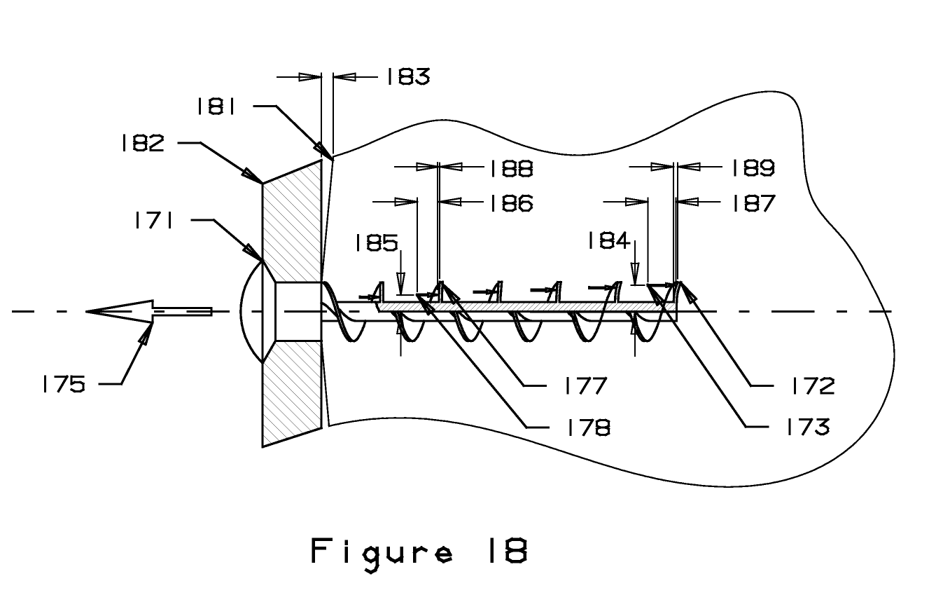

FIG. 18 shows the bone screw (171) of FIG. 17 attaching an appliance (182) to bone (181). The appliance (182) may be any appliance, such as a medical, orthodontic or dental appliance, that needs to be secured to bone. The appliance is loaded by a force vector (175) and is therefore deforming bone (181) an amount (183). Smaller opposite direction force vectors (173 and 178) represent the magnitude (186 and 187) of the forces (173 and 178) and the radial distance (184 and 185) from the center of the bone screw (171) of forces (173 and 178) acting on bone screw threads (172 and 177). Note that the radius (184) of the force vector on bone screw thread (172) is greater than the radius (185) of the force vector on bone screw thread (177). The magnitudes (186 and 187) of these individual force vectors (173 and 178) as represented by the length of the force vector arrows (184 and 185) may be the same or they may be different from each other. In FIG. 18, the magnitude (186 and 187) of these individual force vectors (173 and 178) being different and radius (184) being greater than radius (185) produce a greater deformation (189) of bone screw thread (172) than the deformation (188) of bone screw thread (177). Force magnitude (187) deforming bone screw thread (172) at the distal end of the bone screw (171) would normally be greater than force magnitude (186) deforming bone screw thread (177) toward the head end of the bone screw (171) for a constant cross section bone screw with a thread of equal thickness.

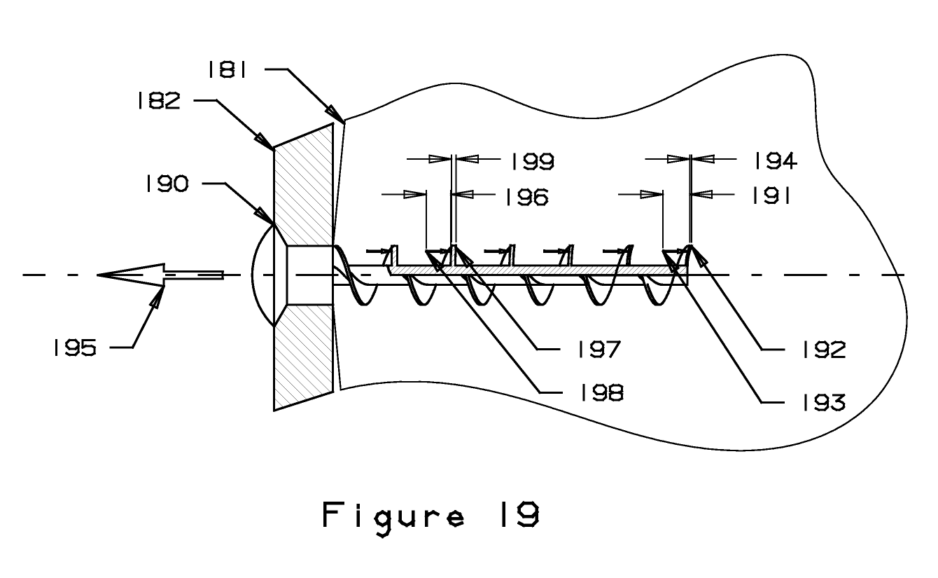

FIG. 19 shows a bone screw (190) with a constant cross section attaching an appliance (182) to bone (181). Bone screw (190) has bone screw threads of different thicknesses (194 and 199). The thickness (194) of the bone screw thread (192) at the distal end is less than the thickness (199) of the bone screw thread (197) that is toward the head end of the bone screw; therefore, thread (192) is more flexible than thread (197). This would allow force (196) to be larger or possibly equal to force (191). Provided that the maximum allowable stress in the bone is substantially uniform, the more equal the force vectors (193 and 198) on the individual bone screw threads (192 and 197) are, the greater the allowable total force (195).

FIGS. 1, 2, 3, 5, 10, 11, and 12 show a screw with a button head and a shoulder. FIGS. 7, 13 and 17-19 show a screw with a countersunk head and a shoulder. FIGS. 9, 14, 15 and 16 show a screw with a countersunk head only. These represent three of the many head attachment configurations that would be matched to the design of the orthopedic apparatus. Any head configuration, including other head configurations known in the art, could be used in any of the bone screw designs of the present invention.

While all of the bone screws illustrated herein have single threads, the same principles of this invention would apply to screws configured with two or more threads.

In another embodiment of the present invention, the bone screw is made of a material which, when formed into a screw, has a modulus of elasticity substantially lower than that of stainless steel or titanium, such that when the material is formed into a screw, the cross-sectional area and modulus of elasticity relationship described above is obtained. With this material, the load is more evenly distributed along a length of the thread of the screw, thus strengthening the screw bone attachment system. In other embodiments, a material with a lower modulus of elasticity than titanium or stainless steel may be used in combination with any of the bone screws shown in the embodiments of FIGS. 2, 4, 5, 7 and 9 to create a relatively even distribution of stress along a length of the bone screw thread to the parent material, by equating the effective cross-sectional area of the screw times its modulus of elasticity to the effective cross-sectional area of the bone times its modulus of elasticity.

Accordingly, it is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A bone screw apparatus comprising a bone screw capable of being attached to a bone, wherein the bone screw comprises:
a) a head;
b) a shoulder connected to the head;
c) a shank extending from the shoulder and having a shoulder end closest to the shoulder and a distal end opposite the shoulder end; and
d) at least one thread attached to the shank;
wherein there is substantially uniform loading along a substantial portion of a length of the thread of the bone screw when the bone screw is attached to the bone;
wherein an effective cross-sectional area of the bone screw times a modulus of elasticity of the bone screw matches an effective cross-sectional area of the bone times a modulus of elasticity of the bone such that the bone screw deforms on a same order as the bone when the bone screw is attached to the bone; and
wherein the thread is attached to the shank at a location and in a configuration selected from the group consisting of:
i) the thread is attached to the shank at the shoulder end of the shank and the thread is free to deform longitudinally relative to the shank between the attachment point and the distal end of the shank;
ii) the thread is attached to the shank at the distal end of the shank and the thread is free to deform longitudinally relative to the shank between the attachment point and the shoulder end of the shank; and
iii) the thread is attached to the shank at the shoulder end of the shank and the distal end of the shank and the thread is free to deform longitudinally relative to the shank between the attachment points at the shoulder end and the distal end of the shank.

2. The apparatus of claim 1, wherein, for at least one point along a length of the bone screw, the effective cross-sectional area of a material of the bone screw times the modulus of elasticity of the material is substantially equal to the effective cross-sectional area of the bone times the modulus of elasticity of the bone.

3. The apparatus of claim 1, wherein a thickness of the thread is substantially less than a height of the thread.

4. The apparatus of claim 1, further comprising an artificial bone material that fills any internal voids in the bone screw.

5. The apparatus of claim 1, wherein the bone screw has a variable cross-sectional area along at least a portion of a length of the bone screw.

6. The apparatus of claim 1, wherein, for at least one section along a length of the bone screw, a spring rate of the section of the bone screw times the length of the section of the bone screw is substantially equal to the effective cross-sectional area of the bone times the modulus of elasticity of the bone.

7. A bone screw apparatus comprising a bone screw capable of being attached to a bone, wherein the bone screw comprises:
a) a head;
b) a shank extending from the head and having a head end closest to the head and a distal end opposite the head end; and
c) at least one thread extending from the head;
wherein there is substantially uniform loading along a substantial portion of a length of the thread of the bone screw when the bone screw is attached to the bone;
wherein an effective cross-sectional area of the bone screw times a modulus of elasticity of the bone screw matches an effective cross-sectional area of the bone times a modulus of elasticity of the bone such that the bone screw deforms on a same order as the bone when the bone screw is attached to the bone; and
wherein the thread is attached to the shank at a location and in a configuration selected from the group consisting of:
i) the thread is attached to the shank at the head end of the shank and the thread is free to deform longitudinally relative to the shank between the attachment point and the distal end of the shank;
ii) the thread is attached to the shank at the distal end of the shank and the thread is free to deform longitudinally relative to the shank between the attachment point and the head end of the shank; and
iii) the thread is attached to the shank at the head end of the shank and the distal end of the shank and the thread is free to deform longitudinally relative to the shank between the attachment points at the head end and the distal end of the shank.

8. The apparatus of claim 7, wherein, for at least one section along a length of the bone screw, a spring rate of the section of the bone screw times the length of the section of the bone screw is substantially equal to the effective cross-sectional area of the bone times the modulus of elasticity of the bone.

9. The apparatus of claim 7, wherein a thickness of the thread is substantially less than a height of the thread.

10. The apparatus of claim 7, wherein, for at least one point along a length of the bone screw, the effective cross-sectional area of a material of the bone screw times the modulus of elasticity of the material is substantially equal to the effective cross-sectional area of the bone times the modulus of elasticity of the bone.

11. The apparatus of claim 7, further comprising an artificial bone material that fills any internal voids in the bone screw.

12. The apparatus of claim 7, wherein the bone screw has a variable cross-sectional area along at least a portion of a length of the bone screw.

13. A method of choosing a bone screw capable of being attached to a bone, comprising the step of designing the bone screw to have an effective cross-sectional area times a modulus of elasticity of the bone screw that matches an effective cross-sectional area of the bone times a modulus of elasticity of the bone such that the bone deforms on a same order as the bone screw when the screw is attached to the bone, wherein the effective cross-sectional area of the bone screw is determined by finite element analysis.

14. The method of claim 13, further comprising the step of creating a catalog of effective cross-sectional areas of bone screws.

15. The method of claim 13, further comprising the step of inserting the bone screw into at least a portion of a bone, wherein the bone screw deforms substantially a same amount as the bone when force is applied to the bone and the bone screw.

16. The method of claim 13, further comprising the step of providing substantially uniform loading along a substantial portion of a length of a thread of the bone screw when the bone screw is inserted into the bone.

17. The method of claim 13, wherein the effective cross-sectional area times the modulus of elasticity of the bone screw is substantially equal to the effective cross-sectional area of the bone times the modulus of elasticity of the bone.
